(19) United States
(12) Patent Application Publication
(10) Pub. No.: US 2026/0020643 A1
(43) Pub. Date: Jan. 22, 2026

(54) DEVICE AND METHOD FOR FOOTWEAR CUSTOMIZATION

(71) Applicant: Superfeet Aggregator LLC, Seattle, WA (US)

(72) Inventor: Edward Ponomarev, Carlsbad, CA (US)

(21) Appl. No.: 19/340,011

(22) Filed: Sep. 25, 2025

Related U.S. Application Data
(63) Continuation of application No. PCT/US2024/025298, filed on Apr. 18, 2024.
(60) Provisional application No. 63/460,121, filed on Apr. 18, 2023.

(51) Int. Cl. A43D 1/02; A43B 13/14; A61B 5/103; B33Y 50/00; B33Y 80/00

(52) U.S. Cl. CPC A43D 1/022; A43B 13/14; A61B 5/1038; B33Y 50/00; B33Y 80/00

(57) ABSTRACT

A device and method for the formation of customized footwear insoles and pads is provided. The system employs computers operatively engaged to scanning components to produce individual electronic footprints of a foot of a wearer. Software operating to the tasks of employing the digital data from one or a plurality of electronic footprints, correlating to scans of the feet of a footwear user, will customize an insole or pad for positioning in the footwear to more comfortably support the foot therein.

### CROSS-REFERENCE TO RELATED APPLICATION(S)

This application is a continuation of International Application No. PCT/US2024/025298, filed on Apr. 18, 2024, which claims priority to U.S. Provisional Patent Application No. 63/460,121, filed on Apr. 18, 2023, the entire contents of both of which are incorporated herein by reference and relied upon.

### TECHNICAL FIELD

The present device relates to footwear, such as shoes and the like. More particularly, the disclosed device and system relate to the determination of optimal shoe sole configurations for users having both body structural and body blood circulation issues which affect their feet.

### BACKGROUND

Peripheral artery disease (PAD) is an abnormal narrowing of arteries other than those that supply the heart or brain. PAD can happen in any blood vessel, but it is more common in the legs and particularly can cause medical issues in the feet. PAD differs from peripheral venous disease in that patients tend to have narrowed or blocked arteries, since it is arteries that carry oxygen-rich blood as it moves away from the heart to other parts of the body. The further oxygen-rich blood travels from the heart and lungs along narrowed arteries, the more severe the medical problems that arise from cells of the body not receiving a sufficient blood supply. Complications of such poor blood flow to patients may include pain, discomfort, and the potential for infection or tissue death, which may require amputation.

The risks of PAD, especially to the feet, are significantly enhanced for patients also having diabetes. A diabetic foot is any pathology that results directly from peripheral artery disease (PAD) and/or sensory neuropathy affecting the feet in diabetes mellitus.

Diabetic foot conditions can be acute and chronic complications of diabetes. The presence of several characteristics of diabetic foot pathologies, such as infection, diabetic foot ulcer, and neuropathic osteoarthropathy, is called diabetic foot syndrome.

In order to minimize the potential for pain and increased injury to the feet of PAD and diabetic patients, the footwear worn should be configured to be comfortable and to help minimize foot problems caused by ill-fitting footwear.

The foregoing background concerning conventional PAD and the risks and limitations related therewith are intended to be illustrative and not exclusive, and they do not imply any limitations on the system and invention described and claimed herein. Various other limitations of the related art of footwear for PAD and diabetic patients are known or will become apparent to those skilled in the art upon a reading and understanding of the specification below and the accompanying drawings.

It is an object of this invention to provide a system for the determination of the optimum fit for footwear for patients suffering from PAD and diabetes, sores, calluses, and from other medical issues of their feet, through the employment of foot scans and 3D customization of insoles based thereon.

It is a further object of this invention to produce footwear insoles which are configured to provide customized pressure and padding to multiple mapped individual areas of the foot of a wearer, based upon the ascertaining of pressure points on the foot of the wearer in a pressure footprint.

It is a further object of this invention to provide such a customized footwear which may also employ temperature scanning of areas of the foot to determine areas of high and low blood circulation and to adapt the customized pressure and padding of manufactured insole and footwear to accommodate such based on an electronic temperature footprint.

It is yet another object of this invention to provide such customized footwear which is configured to help reduce any pain or discomfort of the wearer based on a topographical footprint resulting from a scan of the foot.

It is a further object of this invention to provide a system for scanning of one or both feet of a patient and based on 3-D, weight scans, temperature scans, and topographical scans, producing a highly customized insole configured to accommodate foot health issues and mitigate further such issues.

These and other objects, features, and advantages of the present foot scanning and footwear production system herein, as well as the advantages thereof over existing prior art, which will become apparent from the description to follow, are accomplished by the improvements described in this specification and hereinafter described in the following detailed description which fully discloses the invention, but should not be considered as placing limitations thereon.

### SUMMARY

The present invention employs scanning components to determine pressure points upon the foot of a human and/or areas of high and low blood circulation therein.

By the term scanning component is meant the employment of any electronic, optical, or mechanical pressure sensing component, such as a pressure sensing pad, which measures multiple points of contact for pressure from the bottom surface of a foot. For example only and in no way limiting, such conventional pressure sensing pads or scanners employ one or a combination of force sensors (or pressure sensors), load cells, strain gauges, micro-electrotechnical systems (MEMs), and/or force-sensitive resistors (FSRs) or other force sensing components, and an electronic interface circuit in communication with a microprocessor. The microprocessor employs electronic signals communicated from the pressure sensing components to construct a digital pressure “footprint” of the foot placed upon the mat, which identifies individual areas of high pressure contact, mid pressure contact, and low pressure contact of the support surface with the foot.

The term scanning component may also include singularly or in combination with the pressure sensing pad and a topographical 3D scanner below, a surface temperature sensing component which will capture and form a temperature footprint of areas of the lower surface of a foot and show temperature differences in multiple areas which are indicative of the blood circulation of the foot of the user at the time the temperature is sensed. Such a surface temperature scanning component for example and in no way limiting may be a FLIR imaging device which will produce an image of a foot surface and delineate multiple areas by the temperature of the foot surface in those respective areas. Such is indicative of the amount of blood flow communicated to those areas.

The term scanning component may also include any laser, lidar, light emitting or photographic or other projected light or photographic means which will scan the exterior of a foot and thereafter generate an electronic image of a topographical footprint of the lower surface and/or upper surface and/or side surfaces of the foot of a person. Such 3D scanners, for example, are available from Hewlett Packard in many versions and are available from other manufacturers.

Any or all of the above-noted scanning components and methods may be employed herein singularly or in combination with one or a plurality of the other scanning components. Currently, a combination of the outputs from such scanning components are especially preferred where they may be overlaid upon each other electronically to produce a combination scan footprint. Such a combination scan footprint includes the topographical surfaces of the scanned foot, the temperature areas sensed in the same foot, and weight or pressure sensed areas of the same foot.

Using the produced and communicated imaging or digital combination footprints of each respective foot herein, the system, employing computers and software running in electronic memory operatively engaged with one or a plurality of scanning components, will operate alone or in combination:

use one of the contact or optical scanning electronic images to determine a foot size of the user and relate a footwear base portion and a footwear upper portion engageable to the base, correlating to the determined foot size in an electronic image of a foot size footprint;

determine an exterior topography of the foot of the wearer from scanning foot areas thereon to show projections, sores, calluses, bandage areas, and other projecting areas from the foot in an electronic image topography footprint;

determine areas of differing skin temperature sensed using a temperature scanner, such as a FLIR or a pressure contact scanning device, to generate an electronic footprint of the scanned foot to depict areas or locations in the footprint of areas which have higher or lower temperature in an electronic image of a temperature footprint;

determine areas upon the foot which have higher or lower pressure being communicated to a pressure sensor with the wearer standing thereon to produce depictions or mapped areas of higher and lower pressure and/or image areas showing if the patient has flat feet (pes planus), supination or pronation, in an electronic image or pressure footprint;

electronically overlay two or more of the electronic images provided by the electronic footprints generated and employing one or a combination of overlain footprints including one or more of the foot size footprint, the topography footprint, the temperature footprint, and the pressure footprint to show aligning areas from the multiple electronic footprints and thereby produce an electronic image of a combination footprint;

employ software operating to employ the combination footprint to determine any aligned positions of foot topography, higher temperature areas, higher weight areas and lower weight areas which align, and any un-aligned areas to calculate locations based on the insole to be formed for softer cell support, position for harder cell support, positions for a raised surface area and positions for recesses to accommodate foot structure, and determining a type of material and/or a cell matrix geometry formation of material and optimum configuration of the chosen material, to form one or a plurality of an insole or combined lower padding portions or an insole, which sized and shaped for positioning within the footwear base, in positions determined to provide topographical reliefs and differing durometer contact against respective foot areas;

form the insole in one portion or from a plurality of lower padding portions, in a configuration determined to best provide support foot topographical relief areas, and varying durometer padding to the determined foot areas of the lower foot they contact;

position each formed insole in a footwear base in positions determined to correlate to specific foot area for which each was formed;

optionally, form one or a plurality of upper padding portions to support and form a padded contact against areas of the upper surface of the foot;

position the upper padding portions in operative engagement with the chosen footwear upper portion; and

engage the formed footwear with the foot of the wearer.

Optionally, the system herein may include a pressure sensing pad layer and/or a temperature sensing pad layer on the formed insole. This pressure/temperature sensing layer may be positioned as a layer within the formed insole or above or below it as required. So positioned it will operate to sense temperatures on different areas of the foot of the user in contact with the layer on an ongoing basis to produce a temperature map. Data from such temperature sensing may be captured in electronic memory and/or transmitted to a computing device, wherein the effectiveness of the configured padding and footwear can be determined on an ongoing basis, and based on a database of such temperature maps, can be employed to predict future foot ailments before they happen. Such will allow for subsequent adjustments to the formed padding and positioning thereof. Such a pressure sensing pad may be powered by an onboard battery and transmit sensed temperatures and locations thereof by wireless transmission, such as Bluetooth or WiFi, at predetermined future times to the system provider.

The configuration of the pads or insole and shape thereof will be determined by the system herein in a manner to provide optimal padding, recesses, supporting raised areas, or protection to the foot of the user based on the scanned topography, pressure points, and temperature areas of the produced singular or electronically combined electronic footprints in different areas. The system will operate to customize the shape and configuration of each such pad or insole by manipulating the polymer, elastomer, or plastic or other material type used for printing, shape, and material of the formed unit cells to obviate the pain and discomfort from topographical projections detected such as sores and calluses, and other sense pressure points and other areas of the foot of the user.

In a particularly preferred mode of the system herein, the insoles or foot pads are 3D printed in a layered matrix formed of individual unit cells underlying the top layer or surface layer of the insole. This 3D printing allows the system to micro-form multiple areas of each insole by manipulating the geometry and/or the material of individual formed unit cells in the overall matrix to thereby provide highly customized compression resistance and give, and/or varying durometer and padding of the formed insole, in multiple areas of the insole correlating to sensed topographical and temperature and pressure points identified in the electronic footprint, in a configuration determined to best protect and provide the foot of the user the most comfortable support. By forming more or less connections and cross members in the formed matrix, the durometer may be infinitely adjusted to accommodate that determined best for the patient.

Conventional polymeric or elastomer 3D lattice structures, commonly, are configured with variable design element considerations which is one manner the system herein employs to customize the formed sole and foot pads to the foot of each patient or wearer. The formed geometry of the cells of the printed lattice considers to the physical size and shape of the lattice and individual cells forming it, and the formed pattern is arrayed throughout the structure of the insole or pad. For example and in no way limiting, such formed unit cells can be of any conventional shape and construction, such as Cubic, Rhombicuboctahedron, Octet-truss, Kelvin Cell, BCC (Body Centered Cubic), FCC (Face Centered Cubic), HCP (Hexagonal Close-Packed) and Diamond. Of course, other cell shapes and constructions may be employed and are anticipated. Each such geometry of such unit cells has known stiffness and elasticity and deformation specifications, and thus, can be employed, as needed, to form the customized insole from the material and matrix geometry calculated to yield the insole with the desired parameters for a patient or wearer.

Where the individual 3D printed cells are repeated within the formed layers, such are conventionally known collectively as unit cells. Changing the shape and the cross-linking and thereby the resulting formed cell structure and/or the material forming the unit cells will have a resulting change in the stiffness or durometer of the area of the formed lattice. Thus, a formed sole can be highly customized with individual areas thereof formed of unit cells which are softer, stiffer, or are formed to position recesses on the upper layer to accommodate foot topography.

The stiffness or modulus of the 3D formed lattice refers to the force required to deform the cells and overall structure. This modulus is typically defined for small deformations when the lattice response is fully elastic.

The buckling response of a formed 3D lattice structure, such as the insole or foot pads herein, describes the way that a lattice structure yields under force, such as weight and depends upon the structural instability of lattice elements as they deform. Not all lattice unit cell structures exhibit buckling and buckling is not always a desirable feature.

Another factor which is variable through the formation and positioning of unit cells in the printed sole is energy dissipation. The energy dissipation of a lattice structure refers to its ability to absorb energy while it is being deformed. This too may be adjusted by changing the material employed to print some or all of the unit cells in the lattice structure forming the insole and/or changing the shape and cross-linking of the formed unit cells.

The 3D printed configuration of the insoles herein may operate to form horizontally or vertically disposed spring-like configurations of the formed members, which are cross-linked in the individual unit cells forming the matrix making up the insoles herein. For example, each formed unit cell may be adjusted for diameter of the circular path and diameter of the material forming the elongated flexible members forming a unit cell with customized durometer areas of the insoles or pads.

By adjusting the 3D printer to change the material type or the geometric unit cell type or shape or construction of the unit cells forming the matrix of the insole, a highly customized insole can be created which positions recesses and/or lower durometer areas aligned with sores, higher temperature areas or physical ailments of the foot. Recesses can also be formed in the insole in areas correlating to topographical projections sensed in the topographical footprint or areas where pads or bandages are engaged to the foot. Where customized stiffer areas are desirable, such as with pronation of the feet of the wearer, stiffer areas or raised areas or recessed areas of the formed insole can be positioned with high accuracy to match the physical requirements of each individual foot found in the electronic scans thereof.

The system may also, using a database of polymeric, metal, blastomeric, carbon fiber, and other 3D material matrix structural attributes, may employ material-matching software operating to the task of choosing which available polymeric or blastomeric or other printable material to employ, to form the cell structures of each insole and in each area forming such a pad or insole. Each such customized area will correlate to a contact area with a determined area of the single or combined electronic footprint of the scanned foot of the wearer of the footwear.

This material matching step will allow the employment of printable or extrudeable material for unit cells which each has a varying compressive modulus, cell matrix formation, and tensile strength, as well as a known stiffness. This will allow for the system to match and employ a printing material having a compressive modulus, stiffness, and tensile strength, which, once cured, form interconnected unit cells that work best to support and pad the specific foot of the wearer and multiple areas thereof.

For example and in no way limiting, polymeric materials such as ABS, PLA, polyamide-nylon, and, High Impact Polystyrene or HIPS, or other configurations of polyethylene, have known structural standards when formed to a 3D matrix, which can be held in a material database and matched to the best formation of a pad or pads to support the scanned foot of a user. The system is not limited to plastic or polymeric materials and other materials, such as carbon fiber, and any other material as would be employed by those skilled in the art, such as Resins, Carbon Fiber, Graphite, and Nitinol may be employed alone or in combinations.

With respect to the above description, before explaining at least one preferred embodiment of the customized footwear formation device and system herein in detail, it is to be understood that the invention is not limited in its application to the details of construction and/or to the arrangement of the steps in the system in the following description or illustrated in the drawings. The system for optimum footwear configuration and resulting footwear formation herein described, is capable of other embodiments and of being practiced and carried out in various ways, which will become obvious to those skilled in the art on reading this disclosure. Also, it is to be understood that the phraseology and terminology employed herein are for the purpose of description and should not be regarded as limiting.

As such, those skilled in the art will appreciate that the conception upon which this disclosure is based may readily be utilized as a basis for foot scanning to provide configurations for optimum comfort and medical problem prevention, and to produce the resulting footwear providing such. It is important, therefore, that the claims herein be regarded as including such equivalent construction and methodology for foot scanning and footwear formation insofar as they do not depart from the spirit and scope of the present invention.

As used in the claims to describe the various inventive aspects and embodiments, “comprising” means including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements. The term “substantially”, unless otherwise specifically defined, means plus or minus five percent.

These and other objects, features, and advantages of the foot scanning system and resulting production of the optimum configured footwear for each individual wearer as disclosed herein, as well as the advantages thereof over existing prior art, will become apparent from the description to follow. Such are accomplished by the improvements described in this specification and hereinafter described in the following detailed description which fully discloses the system and footwear herein, but should not be considered as placing limitations thereon.

### BRIEF DESCRIPTION OF THE DRAWINGS

The accompanying drawings, which are incorporated herein and form a part of the specification, illustrate some, but not the only or exclusive examples of embodiments and/or features of the disclosed foot scanning system and the production of individualized optimum-fitting insoles and footwear therefrom. It is intended that the embodiments and Figures disclosed herein are to be considered illustrative of the invention herein, rather than limiting in any fashion.

In the drawings:

FIG. 1 depicts a sensing component configured for the positioning of the foot of a patient or wearer thereon to determine higher and lower weight pressure points and/or hotter and colder areas representing circulation-lacking areas of the foot of the wearer or patient.

FIG. 2 shows the foot of a patient or wearer operatively positioned in a weight bearing contact with the sensing component, such as a weight or pressure scanner and/or temperature scanner.

FIG. 3 shows an example of an optical 3D scanner which produces an electronic image rendering a topographical map of the foot of the wearer which will show the surface contours as well as the location of any protrusions, such as sores, bumps, calluses, bandaged areas, and/or the like.

FIG. 4 depicts an electronic image which may be colored in different areas to show more or less pressured contact of the foot, when standing, or it may be colorized to show hotter and colder areas of the exterior of the foot.

FIG. 5 shows an electronic image from a foot scan employing the electronic scanners for pressure or weight showing a patient with a flat arch and over-pronated foot.

FIG. 6 depicts an electronic image scan from a foot scan using the electronic scanners, such as in FIGS. 1 and 2, wherein the foot weight sensed shows a normal arched foot.

FIG. 7 shows an electronic depiction from a foot scan employing a weight/temperature scanner of FIGS. 1 and 2 wherein a high arch of the user and under pronation of the foot is found.

FIG. 8 shows the exterior lower and upper portions of footwear employable in the system and device herein.

FIG. 9 shows a plurality of lattice structures for insoles and pads produced by the system herein which are configured to minimize pressure against the lower foot of the wearer based on the scanned pressure points and circulation of the foot of the wearer.

FIG. 10 shows a produced lattice structure for customized pads for contact against the upper side of the foot of the user which is configurable in one or more sections to produce minimized pressure against the foot of the user.

FIG. 11 depicts a first side view of the assembled customized shoe or footwear for a user with customized upper pads and lower insoles formed of customized cells in the lattice structure to maximize comfort and minimize medical complications.

FIG. 12 depicts the device produced by the system herein of FIG. 11, from an opposite side view, and shows an optional temperature sensing layer which can be configured to communicate temperatures of areas of the foot of the wearer and communicate such to a remote computing device, such as a smartphone.

FIG. 13 depicts steps in the system to produce the customized insoles and/or pads which are customized to the foot of the wearer based on a plurality of electronic scans.

FIG. 14 shows an example of a unit cell printed or produced by the system herein which is customized as to shape and cross-linking of the members and polymeric or blastomeric material to yield support for areas of the foot of the wearer.

FIG. 15 shows the unit cell of FIG. 14 in a compressed configuration which can be calculated based on the geometric shape and materials forming the unit cell.

FIG. 16 depicts an example of stiffness mapping based on the beam diameter and material of the unit cell and which can be used to customize such based on one or a plurality of electronic scans of the foot of the wearer.

FIG. 17 shows customized insoles and pads which are produced with the system herein and have contact surfaces which may mirror the topographical contour of the foot of the wearer as well as accommodate contact points for projection such as sores and calluses.

FIG. 18 shows a sectional view of an insole, such as that in FIG. 9, wherein the sections are formed in a single unit but have customized areas of stiffness as well as recessing to accommodate the user's foot.

FIG. 19 shows the insole, as in FIG. 18, and depicts a weight and/or temperature sensing pad which may be included to take electronic snapshots of the contact of the foot of the user with the formed insole over time to provide electronic images thereof.

### DETAILED DESCRIPTION

In this description, the directional prepositions of up, upwardly, down, downwardly, front, back, top, upper, bottom, lower, left, right, first, second, and other such terms refer to the system and footwear produced thereby, as it is oriented and appears in the drawings and all such terms are used for convenience only and such are not intended to be limiting or to imply that the footwear formation device has to be used or positioned in any particular orientation.

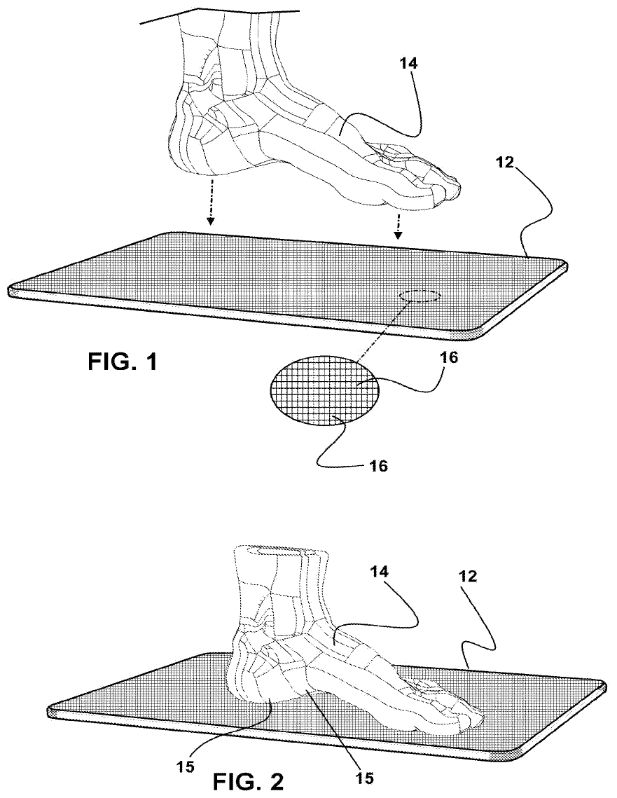

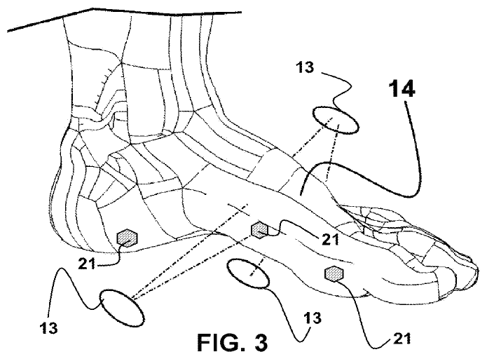

Now referring to drawings in FIGS. 1-19, there is seen in FIG. 1 a depiction of a scanning component 12 configured for measuring the pressure at different areas of the foot 14 of a patient or wearer, and/or temperatures on foot areas, when positioned thereon. Preferably, for pressure points or temperature areas, the measurement is taken in a weight bearing contact, such as in FIG. 2.

As shown in FIG. 1, the pressure or temperature sensing scanning component 12, preferably, has multiple individual sections 16 within the entire grid area of the scanning component 12 which communicate an electronic signal correlating to the pressure of the foot of the user in contact with each section 16. There may be dozens or even thousands of sections 16, each communicating an electronic signal correlating to the pressure of the foot in the respective area contacting the scanning component 12.

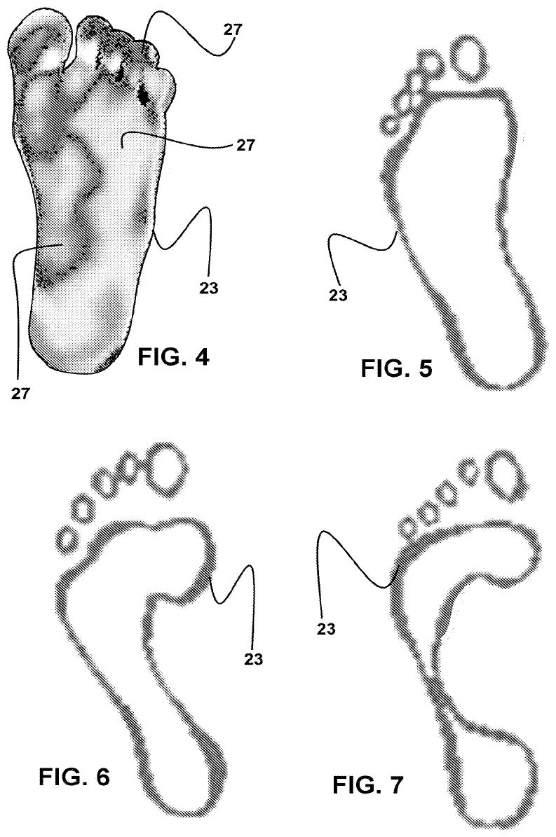

Such a configuration is well known and allows for production of a highly detailed digital image or scan showing colorized areas 15 of the foot which have high or undesired pressure against the scanning component 12. Areas of lower pressure contact may also be determined, such as for ascertaining flat feet or other such issues. Such will provide a highly detailed electronic digital map or image showing areas of the foot 14 needing stiffer or softer support for the foot of the wearer or user based on the force of weight in those areas, such as in FIG. 4, which has differently colorized areas to show more or less pressure contact and in the case of temperature, higher and lower temperature areas.

As noted, this scanning component 12, used in the system herein, may also be or include optical scanners 13, such as laser scanning components, photographic scanning components using digital photography for imaging areas of the foot, and FLIR photography for correlating skin temperature within each scanned area, or other noted scanning components.

The digital imaging output or footprints of the multiple scanners, as noted, may be combined and/or overlain electronically to form a combined digital image or combination footprint of the foot scanned. This combination footprint, as noted, will allow the system to employ software operating for the system to employ the combination footprint to determine the size and positioning and unit cell geometry and construction to yield an optimum configuration of insoles 18 and pads 19 customized for the foot of the wearer.

The insoles 18 formed by the system herein, in shapes and sizes and contours to best contact different areas of the foot 14 of the wearer, may also include upper pads 19 for contact against the upper surface of the foot 14 as well as one or a plurality of sections of the lower positioned insoles 18. The upper pads 19 are configured in shape and size and number, such that they may be engaged with the upper section 20 of the formed footwear in a manner where they contact areas of the foot which were scanned and for which the upper pads 19 were formed to support.

As noted, FIG. 3 shows an example of an optical 3D scanner 13 which employs light, such as lasers, to generate an electronic topographical image rendering a topographical map of the foot 14 of the wearer. Such a topographical image will show the surface contours as well as the location of any protrusions 21 extending from the foot 14. By protrusions is meant herein wounds, sores or bumps or calluses or boney areas or other non-smooth projecting areas of the foot or bandaged areas thereof and the like.

FIG. 4 depicts an electronic image which may be colorized or shaded in different areas 27 to show differing pressures found when standing on the scanning component 12 in the pressure footprint. A similar electronic image is produced for the temperature footprint where it may be colorized to show hotter and colder areas of the exterior of the foot.

Shown in FIG. 5 is another electronic image 23 or pressure footprint from a foot scan employing the electronic scanners for pressure or weight. As shown, it depicts the foot of a patient having a flat arch and over-pronated foot. Such is employable when forming the insole 18 to adjust the unit cell structure of the matrix to include an arched area to support the foot.

FIG. 6 shows an electronic image 23 or pressure footprint from a foot scan using the electronic scanners, such as in FIGS. 1 and 2, wherein the foot weight sensed shows a normal arched foot. Such may be employed during the combination of electronic footprint images by the software operating to form the matrix of the insole 18, to form the top surface with a normal rise in the center area since support is not needed.

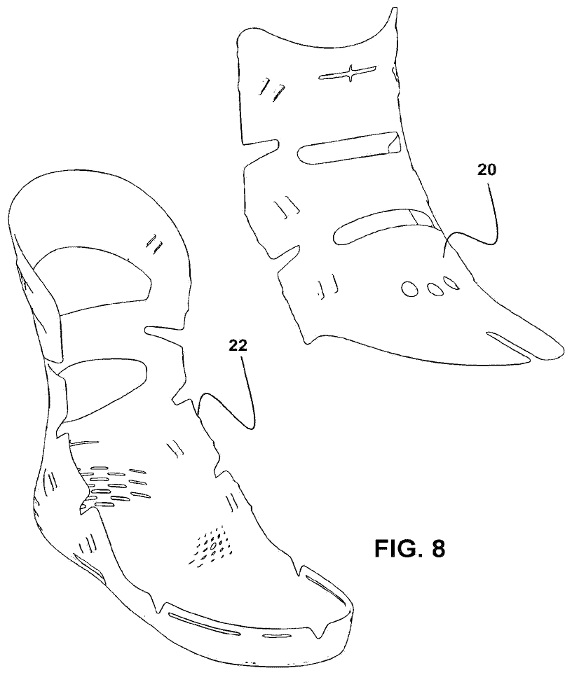

FIG. 7 is another example of an electronic image 23 or pressure footprint from a scan employing a weight/temperature scanner of FIGS. 1 and 2. As shown, there is a high arch of the user and under-pronation of the foot present. In the formation of the insole 18, this may be employed by the software operating to form the matrix and unit cells thereof, to add firmer support to an area of the insole 18 to cause the wearer to properly tilt their feet during walking.

Shown in FIG. 8 are the exterior upper section 20 and the lower section 22 of the formed footwear. It should be noted that the upper section 20 and lower section 22 are configured to engage and form the exterior of the footwear using insoles 18 and pads 19. However, any exterior which will hold at least an insole 18 may be employed, such as for example, a tennis shoe or hiking shoe or other conventional footwear styles. The upper section 20 and lower section 22 may be 3D printed in shapes and shoe sizes determined by the system herein using the scanning components to be best sized and shaped and contoured for the foot 14 of the wearer. They may also be manufactured in advance and used in determined proper sizes and shapes to fit the foot 14 of the wearer, as well as operatively engage and hold the upper pads 19 and lower insoles 18 in the determined proper position to contact determined areas of the foot 14.

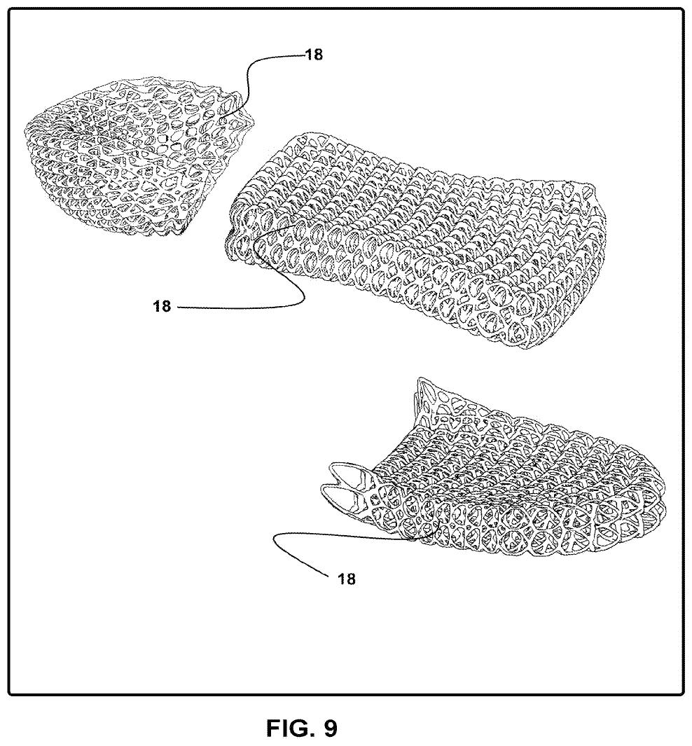

In FIG. 9, an insole 18 which may be formed of a plurality of formed sections of an insole 18. Each section is configured in a size and shape to engage with the chosen lower section 22 of the footwear in positions best determined to contact against matching areas of the foot 14 having more or less pressure exerted thereon or having recesses and raised areas positioned to match protrusions 21 or projecting foot structures such as bunions or calluses. As shown, the pad 18 sections are formed of a 3D printed matrix of individual unit cells having a chosen geometry and stiffness when formed in lattice structures to yield the proper contours, recesses, and stiffness best suited to the foot of the wearer.

As noted above, the system herein, using software running to the task, will employ one or the combination of electronic footprints or scans to determine a shape, pressure areas, topographical configurations, and temperature, from the electronic footprints from one or a combination of the noted scanning components 12 herein to instruct a 3D printer in the forming of the unit cells in the matrix to form each individual insole 18 or pad 19. The compression and spring in multiple areas, the tensile strength, the shape, any recesses or projecting areas in the top surface and other structural considerations will be determined by software on the system running to the task of employing the electronic footprint images alone or preferably in combination, to discern the best respective configuration of an insole 18 and, if employed, pad 19.

Any recesses, higher or raised areas, stiffer lattice areas, softer lattice areas, formed into the insole 18 or pad, will be positioned to mirror the area of the foot 14 requiring such.

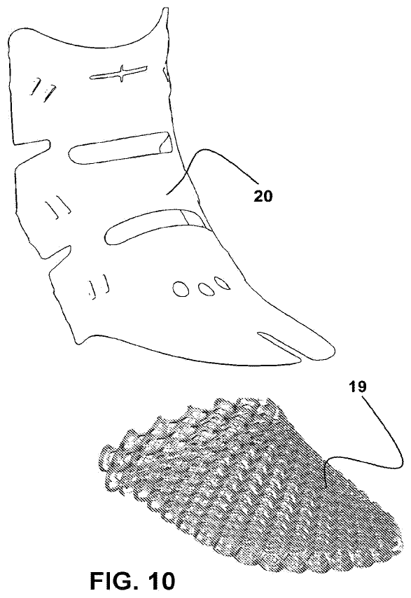

Shown in FIG. 10 is a 3D produced upper located pad 19 configured to engage with the upper section 20 and contact upper areas of the foot 14. As noted, the pads 18 on an upper area may be optional since the insoles 18 are configured to engage within any shoe that might be worn by a user and not just in a formed lower section. As shown, the upper pad 19, much like the insoles 18, is produced with individual unit cells having a chosen cell geometry which when formed to a complete lattice structure configuration, will yield the best determined contact and support in areas matching positions on the top of the foot of the wearer.

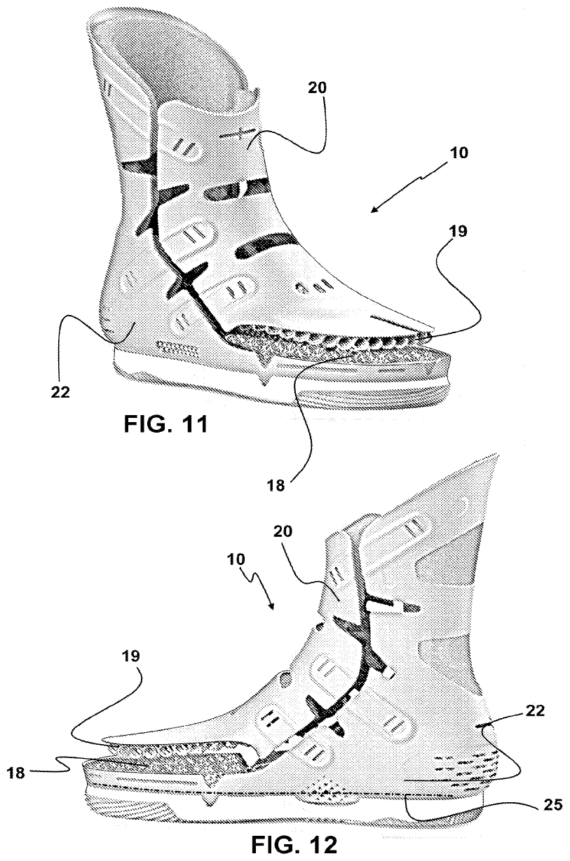

Shown in FIG. 11 is a first side view of an assembled customized shoe or footwear 10 herein. As shown, the footwear 10 includes the customized upper pads 19 and insoles 18 formed by the system herein in a manner to best align with and support the determined areas of the foot 14 they contact. However, the insoles 18 and pads 19 herein may also be used in conventional footwear, such as athletic shoes and dress shoes and any footwear where the formed insole 18 or pad 19 will fit.

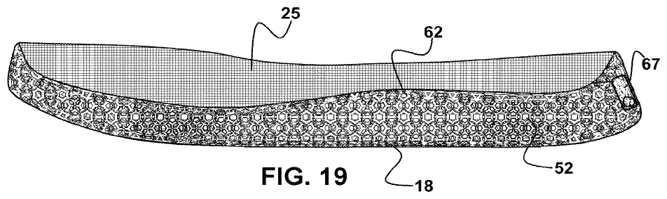

In FIG. 12 is depicted the footwear 10 device produced by the system from an opposite side view. Also shown are an optional weight and/or heat sensing component layer in the form of a pressure and/or temperature sensing portable foot sensing layer 25. When positioned in the finished footwear 10 such as in FIG. 19, with the determined and formed upper pads 19 and lower pads or insoles 18, the foot sensing layer 25, where it is a pressure sensing pad, can communicate the real-time or snapshots of pressure contact of multiple areas of the foot against the lower section 22. This information, as to pressure and/or temperature, can be stored in electronic memory operatively engaged to the sensing layer 25 and later communicated to a computer, such as a smartphone, by wired or wireless communication.

The gathered data, as to ongoing pressure and/or temperature in multiple areas of the foot while wearing the customized footwear 10 and insole 18 may be employed subsequently to modify the shape, size, position, and other configuration elements of the lower pads or insoles 18. This is an important option to include, since it allows for subsequent reconfiguration of the footwear 10 with reconfigured upper pads 19 and insoles 18 to remedy any determined indescribable contact of the foot 14 with the footwear 10.

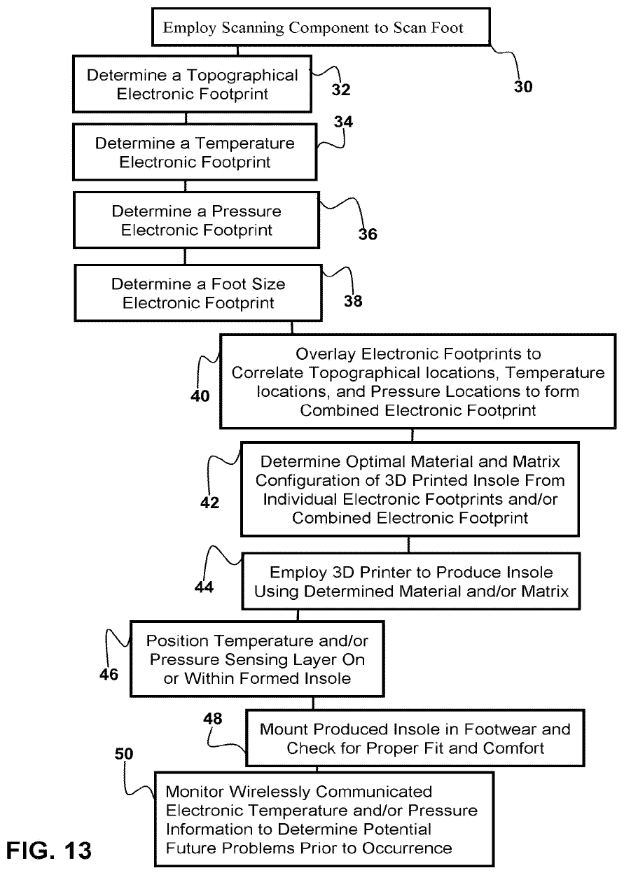

Shown in FIG. 13 are steps in the system employable to produce the customized insoles 18 and/or pads 19 which are customized to match the discerned exterior and pressure areas and temperature areas of the foot 14 of the wearer based on a plurality of electronic scans in the combination.

As noted in all modes of the system and footwear herein, one or preferably a plurality of scanning components are used to scan the foot of the wearer 30. During the scanning process, an electronic scan of the exterior of the foot and contours and projections and recesses thereof may be determined and formed to an electronic topographical footprint 32. Also, using optical or pressure contact scanning of the foot, an electronic temperature footprint may be discerned 34. The scanning process may also include, as noted above, an electronic pressure footprint 36 depicting various areas of the foot 14 having higher or lower pressure contact with support surfaces. Using one or more of the electronic scans, a foot size may be determined 38 which correlates to the intended footwear in which the insole 18 and/or pads 19 will be mounted.

In an especially preferred mode of the system, the electronic footprints may be overlaid electronically to form a combined electronic footprint 40. As noted above, this combined footprint enables software operating to the task of forming the individual unit cells and matrix of the insole 18 or pads 19 to customize each and thereby match it to the wearer.

As noted above, by matching it to the foot of the wearer is meant that the unit cell formation of the matrix, forming the insole 18 or pad 19, is adjusted to provide lower or higher stiffness in area matching foot areas needing such from the pressure footprint recesses or projections from the top surface of the insole 18 or pad 19 in positions matching areas on the foot needing such discerned from the topographical footprint, and stiffness and/or recesses or projection adjustments in areas of the formed insole 18 or pad 19 which will match and contact against areas of the foot of the user requiring such from the temperature footprint. By aligning the multiple electronic footprints using software operating to the task, and aligning requirements for changes to the insole 18 or pad 19 may also be determined.

Employing a plurality of electronic footprints in the combined electronic footprint in the preferred mode of the system, software running in memory on a computer operating to the task of changing individual areas of the matrix forming the insole will determine an optimal material and unit cell geometry 42 to form in each area of the insole 18 or pad 19. This will locate the determined structural requirement at each such area of the insole 18 or pad 19, to match an area of the foot 14 of the user requiring the structural configuration when positioned in contact with the insole 18 or pad 19. Thus, the differing areas positioned to contact against the foot 14 of the wearer can each be customized for stiffness, recesses and projection areas to provide the support to the foot required in that area.

With the unit cell geometries and material for printing determined, a 3D printer is employed to form the matrix 44 to produce an insole 18 or pad 19.

Once the insole 18 or pad 19 is formed to produce customized individual contact areas against the foot 14 of the wearer, optionally, a sensing layer may optionally be engaged with the top surface of the insole 46. The sensing layer is configured with electric power and wireless transmission ability to be able to communicate temperature and/or pressure footprints over time.

With the custom insole 18 and/or pad 19 produced, it may be mounted into the footwear of choice 48. Should the sensing layer have been included, at various times in the future, the output from the sensing layer may be wirelessly communicated 50 to allow the system herein to determine if pressure sensed or temperature sensed in various areas of the insole 18 indicate any changes in configuration should be provided.

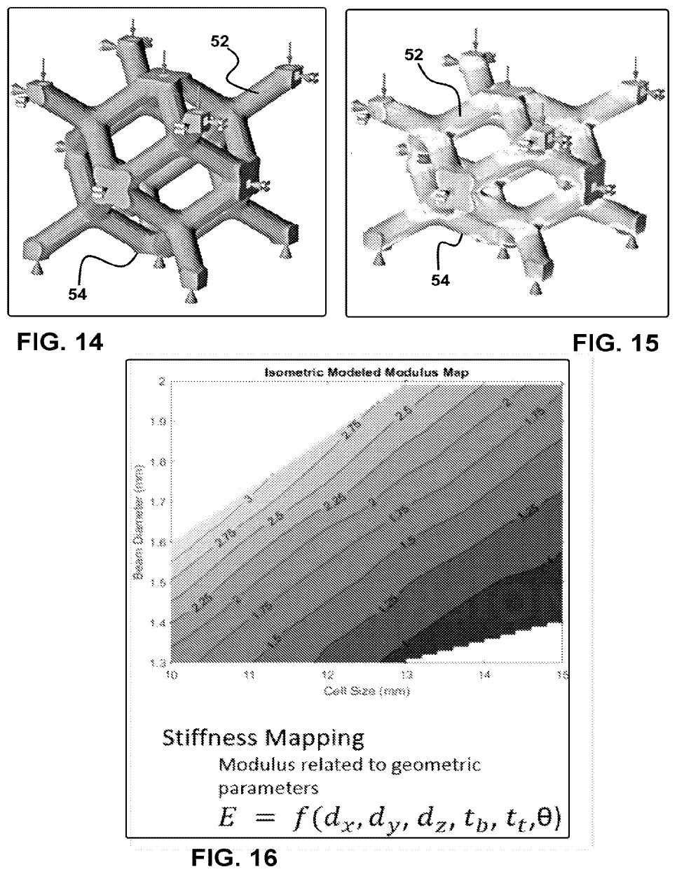

Depicted in FIG. 14 is a unit cell 52 in one geometric shape that may be employed to form a matrix during 3D printing to form the insole 18 or pad 19. As noted above, the shape and geometric configuration of the unit cell 52 may be changed or adjusted to yield the stiffness or compression desired in respective areas of the insole 18 or pad 19 to accommodate the foot of the wearer with customized areas which will mate against individual areas of the foot 14 determined to require such.

FIG. 15 shows the unit cell 52 of FIG. 14 in a compressed state. As noted above, the shape, geometry, cell type, and material used to form the unit cells 52, along with the diameter of the cross members 54 forming it, may all be adjusted to form the unit cells 52 in different areas of the matrix to locate an area thereof adjacent an area of the foot requiring the modification. Stiffness mapping, as shown in FIG. 16, can be used to adjust the material type and the diameter of the cross members 54 or beams to yield the desired durometer or compression for each unit cell 52 in each area of the matrix forming the insole 18 or pad 19 to form that area to contact against the foot and provide the desired support thereto.

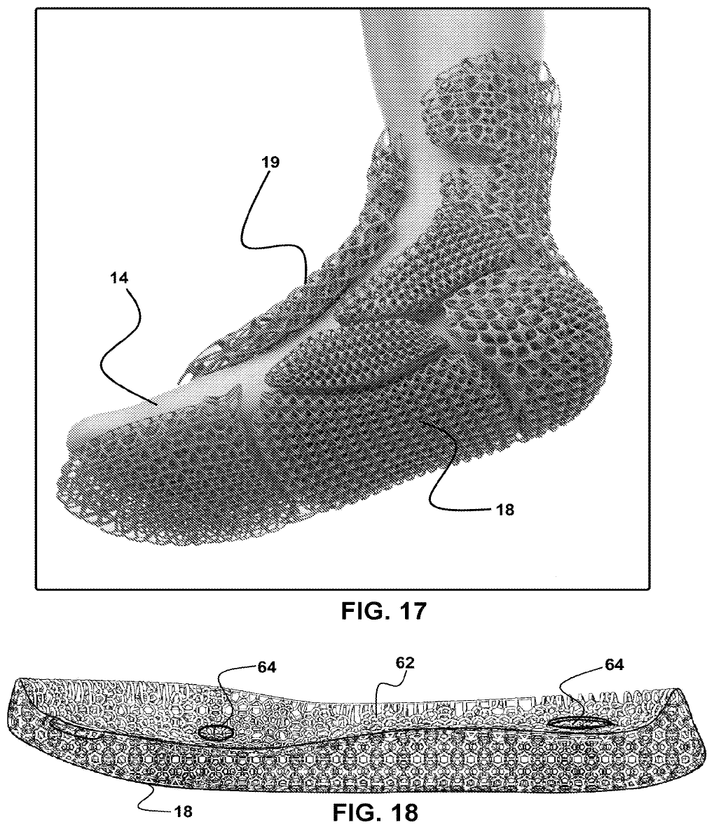

FIG. 17 is an example of customized insoles 18 and pads which are produced with the system herein and have contact surfaces which mirror the topographical contour of the foot of the wearer and are positioned to accommodate contact points for projection such as sores and calluses or recesses or areas of stiffer or softer surfacing. As can be seen, the insole 18 is formed in multiple sections but could be a single piece. Also viewable is the printed matrix of unit cells, formed in differing shapes and geometries, to provide the respective relief areas or support areas determined from the multiple scans.

In FIG. 18 is an insole 18 in half section view through a typical one-piece insole 18 which shows the unit cells forming the matrix which is 3D printed to form the custom insole 18. Also shown are the curving contour 62 and recesses 64 positioned on the top surface of the insole 18 which will align with and contact around projections or protrusions 21 determined from the electronic topographical footprint.

Finally, in FIG. 19 is depicted the insole 18, as in FIG. 18, where the weight and/or temperature sensing layer 25 is positioned on the top surface of the insole 18. As noted, the sensing layer 25 may be included to take electronic snapshots of the contact of the foot of the user with the formed insole over time to provide electronic images thereof. A wireless transmitter 67 has an onboard power supply, such as a battery, and is operatively connected to the sensing layer 25 to thereby capture and transmit electronic images showing one or both of pressure locations and temperature zones which form during wearing of the footwear.

As noted, any of the different configurations and components of the footwear shown and described herein, or the steps in determining the optimum construction thereof, can be employed with any other configuration or component shown and described. Additionally, while the disclosed system for optimum configuration of footwear for users and production thereof has been described herein with reference to particular embodiments thereof and components thereof operatively engaged for operation, a latitude of equivalent modifications, various changes and substitutions are intended in the foregoing disclosures and it will be appreciated that in some instance some features, or configurations, or operations of the invention could be employed without a corresponding use of other features without departing from the scope of the invention as set forth in the following claims. All such changes, alternations and modifications as would occur to those skilled in the art subsequent to reviewing this specification, are considered to be within the scope of this invention as broadly defined in the appended claims.

Further, the purpose of any abstract of this specification is to enable the U.S. Patent and Trademark Office, the public generally, and especially the scientists, engineers, and practitioners in the art who are not familiar with patent or legal terms or phraseology, to determine quickly from a cursory inspection the nature and essence of the technical disclosure of the application. Any such abstract included herein is neither intended to define the invention of the application, which is measured by the claims, nor is it intended to be limiting, as to the scope of the invention in any way.

1-7. (canceled)

8. A method for producing custom-formed sole portions insertable into footwear comprising:
capturing a weight-bearing scan of a foot of a wearer while standing upon a first scanning component;
generating an electronic pressure footprint depicting multiple individual pressure areas of the foot having a differing pressure contact thereof upon the first scanning component;
determining a required stiffness of sole portion contact areas to support the pressure areas and to determine a required positioning of the contact areas upon the sole portions to support the individual pressure areas using the electronic pressure footprint; and
forming the sole portions, the sole portions comprising a plurality of unit cells forming a matrix having a top surface for positioning the foot thereon with the contact areas supporting the individual pressure areas using a 3D printer, wherein the sole portions are configured to be inserted within a footwear body such that the foot of the wearer wearing the footwear is supported atop the sole portion with the contact areas supporting the pressure areas.

9. The method of claim 8, further comprising:
capturing an electronic temperature footprint of the foot using a temperature scanner, the temperature footprint depicting different areas of the foot having differing temperatures therein; and
determining a required stiffness of secondary sole portion contact areas to support the different areas of the foot having the differing temperatures using the electronic temperature footprint, wherein the wearer wearing the footwear is supported atop the sole portions with the secondary sole portion contact areas supporting the areas of the foot having differing temperatures.

10. The method of claim 8, further comprising:
employing a second scanning component to capture an electronic topographical footprint of an exterior surface of the foot; and
determining projections and recesses to form on the top surface in positions thereon which are complimentary to contours and protrusions depicted in the electronic topographical footprint using the electronic topographical footprint, wherein the foot of the wearer wearing the footwear is supported atop the sole portions with the projections surrounded by the recesses and complimentary sole portion contours surrounding any of the protrusions.

11. The method of claim 10, further comprising:
capturing an electronic temperature footprint of the foot using a temperature scanner, the temperature footprint depicting different areas of the foot having differing temperatures therein; and
determining a required stiffness of secondary sole portion contact areas to support the different areas of the foot having the differing temperatures using the electronic temperature footprint, wherein the wearer wearing the footwear is supported atop the sole portion with the secondary sole portion contact areas supporting the areas of the foot having differing temperatures.

12. The method of claim 10, further comprising:
positioning a sensing layer on the top surface, the sensing layer configured to capture and transmit electronic images showing one or both of pressure locations on the foot of the wearer and temperature zones on the foot of the wearer occurring during wearing of the footwear; and
communicating electronic images of the temperature zones and the pressure locations to a remote computer for review using a wireless transmitter operatively engaged to the sensing layer.

13. The method of claim 12, further comprising:
generating an electronic combination footprint from an electronic overlay of the electronic images of the pressure locations, the electronic images of the temperature zones, and the electronic topographical footprint;
determining alignments of the contact areas, the projections and recesses, and the different areas of the foot having differing temperatures using the electronic combination footprint; and
forming the sole portions with the matrix supporting the alignments using the 3D printer.

14. A formed sole portion for positioning with a footwear body, the formed sole portion comprising:
a sole portion body formed of a 3D printed matrix of engaged unit cells;
a first geometry of some of the unit cells configured to provide first areas of support on a top surface of the sole portion body; and
a second geometry of some of the unit cells configured to provide second areas of support on a top surface of the sole portion body,
wherein the top surface of the sole portion at the first areas of support is stiffer than the top surface of the sole portion at the second areas of support, and
wherein a foot of a wearer wearing the footwear having the sole portion therein is supported atop the sole portion with first contact areas supporting first areas of the foot determined to require stiffer support and second contact areas of support supporting second areas of the foot determined to require softer support than the first areas of the foot.

15. The formed sole portion of claim 14 wherein the sole portion body further comprises a plurality of pressure sensors configured to detect pressure distribution during gait.

16. The formed sole portion of claim 14 wherein the sole portion body further comprises a plurality of temperature sensors embedded in the 3D printed matrix configured to monitor temperature variations of the foot.

17. The formed sole portion of claim 14 wherein the sole portion body further comprises:
a sensing layer configured to capture pressure data and/or temperature data; and
an electronic memory coupled to the sensing layer and configured to communicate the pressure data and/or temperature data to a separate device communicatively coupled thereto.

18. A method comprising:
receiving, from a scanning component, an electronic scan of an exterior of a foot positioned on a scanning component;
generating an electronic topographical footprint of the exterior of the foot based on the electronic scan;
determining a type of material and a configuration of the material for a custom-formed sole portion based on the electronic topographical footprint; and
generating instructions for forming the custom-formed sole portion based on the determined type of material and the configuration of the material, wherein the custom-formed sole portion comprises a plurality of cells forming a lattice structure having a top surface for positioning the foot thereon, and wherein the custom-formed sole portion is configured to be inserted within a body of footwear such that, when the footwear is worn, the foot is supported by the custom-formed sole portion.

19. The method of claim 18, further comprising:
receiving, from the scanning component, an electronic signal related to a pressure of the foot in contact with the scanning component.

20. The method of claim 18, further comprising:
generating instructions for positioning at least one pressure sensor on a portion of the custom-formed sole portion, wherein the at least one pressure sensor is configured to detect pressure distribution during a gait of the foot.

21. The method of claim 20, further comprising:
receiving, from the at least one pressure sensor, an electronic signal related to a pressure of the foot supported by the custom-formed sole portion.

22. The method of claim 18, further comprising:
generating instructions for positioning a plurality of temperature sensors in the lattice structure, wherein the plurality of temperature sensors are configured to monitor temperature variations of the foot.

23. The method of claim 18, further comprising:
receiving, from the scanning component, an electronic temperature footprint of the foot, wherein the electronic temperature footprint depicts different areas of the foot having differing temperatures therein; and
determining a stiffness of the custom-formed sole portion based on the electronic temperature footprint.

24. The method of claim 18, further comprising:
determining projections and recesses to form on the top surface that are complementary to contours and protrusions depicted in the electronic topographical footprint.

25. The method of claim 24, further comprising:
receiving, from the scanning component, an electronic temperature footprint of the foot, the electronic temperature footprint depicting different areas of the foot having differing temperatures therein; and
determining a stiffness of the custom-formed sole portion to support the different areas of the foot having different temperatures based on the electronic temperature footprint.

26. The method of claim 18, further comprising:
receiving, from a sensing layer positioned on the top surface, electronic images showing one or both of pressure locations on the foot and temperature zones on the foot when the footwear is worn via a wireless transmitter.

27. The method of claim 26, further comprising:
generating an electronic combination footprint from an electronic overlay of the electronic images of the pressure locations, the electronic images of the temperature zones, and/or the electronic topographical footprint;
determining alignments of contact areas, projections, recesses, and different areas of the foot having differing temperatures using the electronic combination footprint; and
sending instructions to a 3D printer to form the custom-formed sole portion with the lattice structure supporting the alignments.
